(19) United States
(12) Patent Application Publication
(10) Pub. No.: US 2026/0232464 A1
(43) Pub. Date: Aug. 13, 2026

(54) FLOW MODIFYING IMPLANT FOR VENOUS OR PERIPHERAL ARTERIAL DISEASE

(71) Applicant: KONINKLIJKE PHILIPS N.V., EINDHOVEN (NL)

(72) Inventors: Megan Ann Miezwa, ROGERS, MN (US); Elodie Delassus, UTRECHT (NL); Christopher Olig, MAPLE GROVE, MN (US)

(21) Appl. No.: 19/148,111

(22) PCT Filed: Jan. 5, 2024
(86) PCT No.: PCT/EP2024/050205

§ 371 Date: Jul. 15, 2025

Related U.S. Application Data
(60) Provisional application No. 63/439,322, filed on Jan. 17, 2023.

(51) Int. Cl. A61F 2/88; A61F 2/06; A61F 2/24

(52) U.S. Cl. CPC A61F 2/88; A61F 2/2476; A61F 2002/068; A61F 2230/0091

(57) ABSTRACT

An apparatus is described that includes a helical flow modifier adapted to be disposed into a lumen. The helical flow modifier alters a flow characteristic of a fluid in the lumen along a helical trajectory. In addition, a method of delivering an apparatus is described. The method comprises disposing a helical flow modifier into a lumen, wherein the helical flow modifier alters a flow characteristic of a fluid in the lumen along a helical trajectory.

### BACKGROUND

Venous thromboembolism, which includes deep venous thrombosis (DVT), is a major contributor to the global disease burden and is the third most common cardiovascular pathology after coronary artery disease and stroke. Lower extremity DVT (LEDVT) can block the venous lumen and leads to venous congestion, swelling, and lower extremity venous valve function damage, resulting in post-thrombotic syndrome (PTS).

Damaged or diseased veins and arteries do not foster laminar flow of blood therein. The turbulent flow that results in these diseased veins and arteries is reduces the free-flow of blood through the body and can result in serious damage to parts of the body.

What are needed are an apparatus and a method for deploying the apparatus that improves venous and arterial flow in damaged or diseased veins and arteries.

### SUMMARY

According to one aspect of the present disclosure, an apparatus comprises: a helical flow modifier adapted to be disposed into a lumen. The helical flow modifier alters a flow characteristic of a fluid in the lumen along a helical trajectory.

According to another aspect of the present disclosure, a method of delivering an apparatus is disclosed. The method comprises disposing a helical flow modifier into a lumen, wherein the helical flow modifier alters a flow characteristic of a fluid in the lumen along a helical trajectory.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A is a perspective view of a helical flow modifier in accordance with a representative embodiment.

FIG. 1B is a perspective view of the helical flow modifier of FIG. 1A showing the direction of fluid flow in accordance with a representative embodiment.

FIG. 1C is a perspective view (shown in cut-away) of the helical flow modifier

FIG. 1D is a top view of the helical flow modifier of FIG. 1A looking in the −y direction and in the y-z plane according to the coordinate system of FIG. 1A.

FIG. 1E is a cross-sectional view of the helical flow modifier loaded in a catheter for deployment in a lumen of a body.

FIG. 2A is a perspective view of a helical flow modifier in accordance with a representative embodiment.

FIG. 2B is a perspective view of the helical flow modifier of FIG. 2A showing the direction of fluid flow in accordance with a representative embodiment.

FIG. 2C is perspective view (shown in cut-away) of the helical flow modifier of FIG. 2A.

FIG. 2D is a top view of the helical flow modifier of FIG. 2A looking in the −y direction and in the y-z plane according to the coordinate system of FIG. 2A.

FIG. 3A is a perspective view of a helical flow modifier in accordance with a representative embodiment.

FIG. 3B is a perspective view of the helical flow modifier of FIG. 3A showing the direction of fluid flow in accordance with a representative embodiment.

FIG. 3C is a perspective view (shown in cut-away) of the helical flow modifier along line of FIG. 3A.

FIG. 3D is a top view of the helical flow modifier of FIG. 3A looking in the −y direction and in the y-z plane according to the coordinate system of FIG. 3A.

FIG. 4A is a perspective view of a helical flow modifier in accordance with a representative embodiment.

FIG. 4B is a perspective view of the helical flow modifier of FIG. 4A deployed in a lumen (shown in cut away) illustrating the direction of fluid flow in accordance with a representative embodiment.

FIG. 4C is a top view of the helical flow modifier of FIG. 4A looking in the −y direction and in the y-z plane according to the coordinate system of FIG. 4A.

FIG. 4D is a perspective view of the helical flow modifier showing a shape of the helical structure designed to resist retrograde flow (in the opposite direction of flow shown in FIG. 4B) in accordance with a representative embodiment.

FIG. 5A is a perspective view of a helical flow modifier comprising a leaflet valve in accordance with a representative embodiment.

FIG. 5B is a perspective view of the helical flow modifier of FIG. 5A illustrating the direction of fluid flow in accordance with a representative embodiment.

FIG. 5C is a perspective view (shown in cut-away) of the helical flow modifier of FIG. 5A.

FIG. 5D is a top view of the helical flow modifier having a monocuspid valve in an open state.

FIG. 5E is a perspective view (shown in cut-away) of the helical flow modifier having a monocuspid valve in an open state.

FIG. 5F is a perspective view (shown in cut-away) of the helical flow modifier having the monocuspid valve in a closed state.

FIG. 6A is a perspective view of a helical flow modifier comprising a bicuspid valve in accordance with a representative embodiment.

FIG. 6B is a perspective view of the helical flow modifier of FIG. 6A deployed in a lumen (shown in cut away) illustrating the direction of fluid flow in accordance with a representative embodiment.

FIG. 6C is a perspective view (shown in cut-away) of the helical flow modifier of FIG. 6A.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

As used in the specification and appended claims, and in addition to their ordinary meanings, the term “approximately” mean to with acceptable limits or degree. For example, “memory wires are heated to approximately the same temperature” means one of ordinary skill in the art would consider the temperatures of memory wires are the same within reasonable measure.

As used in the specification and appended claims, in addition to their ordinary meanings, the term ‘substantially’ means within acceptable limits or degree. For example, the “plurality of first memory wires is substantially the same” means one of ordinary skill in the art would consider the first memory wires to be the same.

Unless otherwise noted, when an element or component is said to be “connected to”, or “coupled to another element or component,” it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present teachings relate generally to improving fluid (e.g., blood) flow by recanalizing vessels of the deep venous system or peripheral arteries by providing a helical flow modifier to foster helical flow in veins and arteries when deployed therein. One such flow pattern in the heart and arterial system is substantially helical flow. Contemporary imaging techniques have demonstrated existence of substantially helical flow in normal arteries. Both in vivo and ex vivo studies and numerical simulations suggest that substantially helical flow limits flow instability and represents natural optimization of fluid transport in the cardiovascular system. Anatomical features of the veins of lower extremities, such as diameter changes, curvatures, and junctions with tributaries, are expected to produce angular momentum on the fluid elements, therefore introducing substantially helical flow. While not wishing to be bound to theory, it is believed that shear stresses substantially helical flow imposes on the endothelial lining, which are very responsive to mechanical stresses that are placed on the inner wall of the artery or vein. By contrast, in regions of venous or arterial disease, the helical laminar flow can be interrupted resulting in pathological conditions. Further details of substantially helical flow in normal veins and arteries are disclosed, for example, in “On the Existence of Helical Flow in Veins of the Lower Extremities” to Lurie, et al. (Journal of Vascular Surgery: Venous and Lymphatic Disorders, April 2013.

In accordance with various representative embodiments, an apparatus and a method are directed to reintroducing helical (“normal”) flow in a damaged vessel. The helical flow modifiers of various representative embodiments beneficially recanalize veins and arteries, and/or improve flow characteristics in the inflow vessels when iliac venous stenting is performed, or are used to manage peripheral artery disease, particularly below the knee where there are limited solutions to maintain patency in critical limb ischemia patients. Generally, the helical flow modifiers of the present teachings foster substantially helical flow of fluid by providing a shape to the vein or artery that promotes substantially helical flow, or by providing a shaped insert that guides fluid flow in a helical manner. Among other features, the substantially helical flow modifiers of the various representative embodiments represent an improvement in the field of medical devices by treating inflow vessels in deep venous disease providing support to iliac venous stent patency, or are designed to treat peripheral arterial disease by promoting helical laminar flow in the diseased artery, or both.

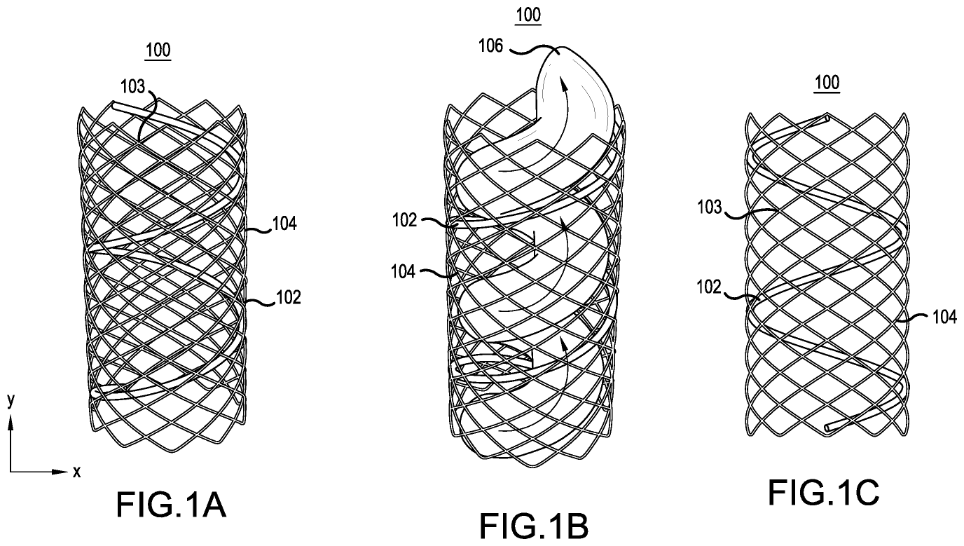

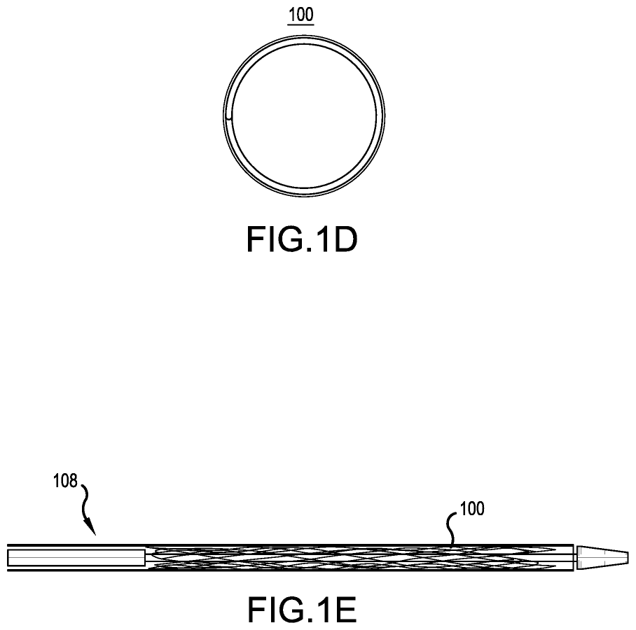

FIG. 1A is a perspective view of a helical flow modifier 100 in accordance with a representative embodiment.

The helical flow modifier 100 is adapted to be disposed into a lumen (not shown in FIG. 1A), as is adapted to alter a flow characteristic of a fluid in the lumen along a helical trajectory. In the representative embodiment of FIG. 1A, the helical flow modifier 100 comprises a helically shaped element 102 disposed in a stent 104 adapted to be disposed in the lumen. In one representative embodiment, the helically shaped element 102 is attached to an inner surface 103 (alternatively referred to as an inner wall) of the stent 104. Alternatively, the helically shaped element 102 may be interwoven in the braid of the stent 104 to alter the shape of the stent 104 to foster helical blood flow as described more fully below. Illustratively, the helically shaped element 102 is fastened to the braid of the stent 104 by one of a number of known methods, including laser welding (e.g., at its ends) or, as noted above, by being woven into the braid of the stent 104.

In this representative embodiment, the helically shaped element 102 comprises nitinol or other known biocompatible material. As described more fully below, the helical flow modifier 100 is deployed in a vein or artery with a catheter or similar device in an initially compressed state. Upon deployment to a desired location, the catheter is removed. The helically shaped element 102 is designed to expand to it original shape and dimension and is force-fit against the inner surface 103 of the stent 104. As such, the helically shaped element 102 has dimensions selected to provide an outward radial (x-direction in the coordinate system of FIG. 1A) force to the inner surface 103 of the stent 104, and therefore to the inner surface of the portion of the vein or artery in which the helical flow modifier 100 is deployed.

Notably, once deployed the helically shaped element 102 provides a helical skeleton or support along the inner surface 103 of the stent 104. This helical skeleton or support in turn provides a shape to the region of the vein or artery in which the helical flow modifier 100 is deployed that promotes substantially helical flow. Alternatively, as noted above, the helically shaped element 102 may be interwoven in the braid of the stent 104 to alter the shape of the stent 104 to foster helical blood flow as described more fully below.

Illustratively, when deployed in the vein or artery, the helically shaped element 102 provides a helical shape to the vein or artery in which the helical flow modifier 100 is deployed, resulting in substantially helical flow through the artery or vein where the helical flow modifier is deployed. Beneficially, the helical flow modifier 100 is deployed in a diseased or otherwise damaged portion of the vein or artery in which it is deployed.

In accordance with a representative embodiment, the helically shaped element 102 comprises a wire having a cylindrical cross section having dimensions selected to provide the force-fit and shape to alter the flow of fluid through the damaged portion of the vein or artery where it is deployed. Beneficially, the helically shaped element 102 has a cross-sectional diameter that is greater than the cross-sectional area of the braid of the stent 104. Beneficially, the helically shaped element 102 provides a comparatively increased outward radial force along its shape against the scaffold of the stent 104, effectively shaping the stent 104 to promote helical flow.

FIG. 1B is a perspective view of the helical flow modifier 100 of FIG. 1A showing the direction of fluid flow in accordance with a representative embodiment. Various aspects and details of the helical flow modifier 100 described in connection with FIG. 1A are of course common to the description of FIG. 1B, and may not be repeated in order to avoid obscuring the description of FIG. 1B.

As shown, the helical flow modifier 100 alters the shape of the vein or artery in the region it is deployed, beneficially resulting in substantially helical flow of fluid 106 through the damaged portion of the vein or artery. Accordingly, the helical flow modifier 100 recanalizes the vessels of the deep venous system or peripheral arteries to the foster substantially helical flow in damaged or diseased portions of the veins and arteries when deployed therein. As such, and as noted above, this substantially helical flow of fluid 106 resembles the normal substantially helical flow of fluid in undamaged diseased (i.e., “normal”) veins and arteries. As such, and like in normal arteries and veins, the resultant substantially helical flow limits flow instability and represents natural optimization of fluid transport in the cardiovascular system.

FIG. 1C is a perspective view (shown in cut-away) of the helical flow modifier 100 of FIG. 1A of the helical flow modifier 100 along line 1C-1C of FIG. 1A. Various aspects and details of the helical flow modifier 100 described in connection with FIGS. 1A and 1B are of course common to the description of FIG. 1C, and may not be repeated in order to avoid obscuring the description of FIG. 1C.

Again, the helical flow modifier 100 comprises a helically shaped element 102 disposed in a stent 104 adapted to be disposed in the lumen. As noted above, the helically shaped element 102 is attached to the inner surface 103 (in this case in the rear of the stent 104) inner wall) of the stent 104. As described more fully below, the helical flow modifier 100 is deployed in a vein or artery with a catheter or similar device in an initially compressed state. Upon deployment to a desired location, the catheter is removed. The helically shaped element 102 is designed to expand to it original shape and dimension and is force-fit against the inner surface 103 of the stent. As such, the helically shaped element 102 has dimensions selected to provide an outward radial (x-direction in the coordinate system of FIG. 1A) force to the inner surface 103 of the stent 104, and therefore to the inner surface of the portion of the vein or artery in which the helical flow modifier 100 is deployed.

FIG. 1D is a top view of the helical flow modifier of FIG. 1A looking in the-y direction and in the y-z plane according to the coordinate system of FIG. 1A. [

Notably, FIG. 1D illustrates there is an open flow lumen. When there is no alteration of the shape of the stent from the wire, then the flow will be affected via the mechanism of a shaped insert, as described above. When the wire is configured such that it shapes the braided scaffold, then the mechanism would be due to the re-shaping of the vein, again, as noted above.

FIG. 1E is a cross-sectional view of the helical flow modifier 100 loaded in a catheter 108 for deployment in a lumen of a body. Various aspects and details of the helical flow modifier 100 described in connection with FIGS. 1A-1D are of course common to the description of FIG. 1E, and may not be repeated in order to avoid obscuring the description of FIG. 1E.

As shown in FIG. 1E, the helical flow modifier 100 is deployed the catheter 108 (or similar device in an initially compressed state. Upon deployment to a desired location, the catheter 108 is removed.

The helical flow modifier 100 may be expanded once in the appropriate location in the lumen using a known balloon. For example, stents comprising nitinol generally require post-dilation (e.g., with balloon), to ensure complete and uniform expansion within the vessel to realize the desired shape as described more fully herein.

In the presently described representative embodiment, the helically shaped element 102 is designed to expand to it original shape and dimension and is force-fit against the inner surface 103 of the stent. Alternatively, in embodiments in which the helically shaped element 102 is interwoven in the stent, when deployed, the stent 104 expands to its original shape and dimension. As such, the helically shaped element 102 has dimensions selected to provide an outward radial (x-direction in the coordinate system of FIG. 1A) force to the inner surface 103 of the stent 104, and therefore to the inner surface of the portion of the vein or artery in which the helical flow modifier 100 is deployed.

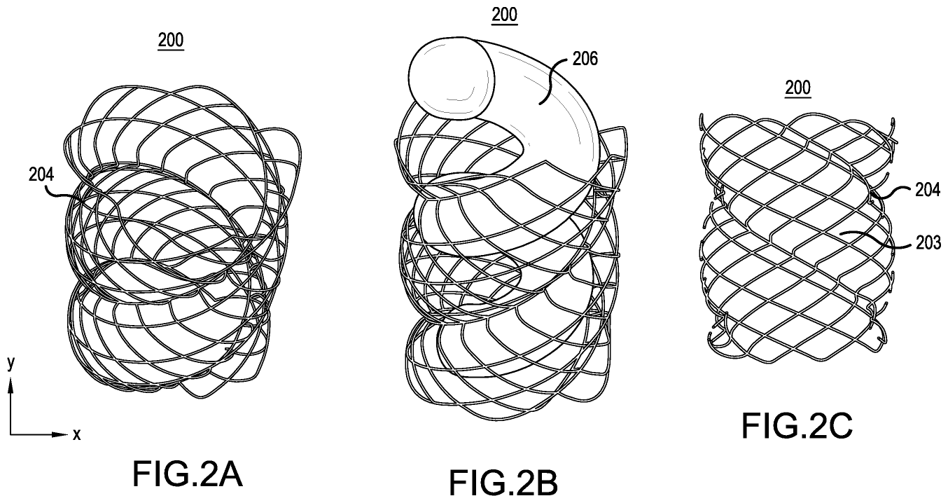

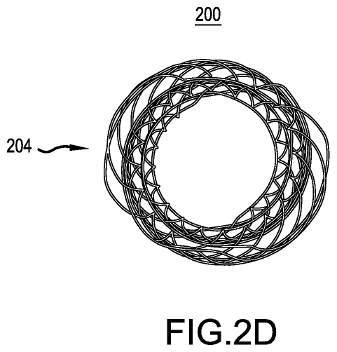

FIG. 2A is a perspective view of a helical flow modifier 200 in accordance with a representative embodiment. Various aspects and details of the various representative embodiments described in connection with FIGS. 1A-1E are common to the description of FIG. 2A, and may not be repeated in order to avoid obscuring the description of FIG. 2A.

The helical flow modifier 200 is adapted to be disposed into a lumen (not shown in FIG. 2A), as is adapted to alter a flow characteristic of a fluid in the lumen along a helical trajectory.

In the representative embodiment of FIG. 2A, the helical flow modifier 200 comprises a helically shaped stent 204 adapted to be disposed in the lumen. In one representative embodiment, the helical flow modifier 200 is attached to an inner surface (alternatively referred to as an inner wall) of the vein or artery in which it is deployed. In this representative embodiment, the helical flow modifier 200 comprises nitinol or other similar shape memory metal or other material. Like the helical flow modifier 100 described above, the helical flow modifier 200 is deployed in a vein or artery with a catheter or similar device in an initially compressed state. Upon deployment to a desired location, the catheter is removed. The helical flow modifier 200 is designed to expand to it original shape and dimension and is force-fit against the inner surface vein or artery. As such, the helical flow modifier 200 has dimensions selected to provide an outward radial (x-direction in the coordinate system of FIG. 2A) force to the inner surface of the portion of the vein or artery in which the helical flow modifier 200 is deployed.

Notably, the helical flow modifier 200 has a substantially helical shape], which is more clearly shown in FIGS. 2B and 2C) and provides a helical support along the inner surface portion of the vein or artery in which the helical flow modifier 200 is deployed. This helical support in turn provides a shape to the inner surface of the region of the vein or artery in which the helical flow modifier 200 is deployed that promotes substantially helical flow. As such, when deployed in the portion of vein or artery, the helical flow modifier 200 provides a shape to the vein or artery in which the helical flow modifier 200 is deployed, resulting in substantially helical flow through the artery or vein where the helical flow modifier is deployed. Beneficially, the helical flow modifier 200 is deployed in a diseased or otherwise damaged portion of the vein or artery in which it is deployed.

FIG. 2B is a perspective view of the helical flow modifier 200 of FIG. 2A showing the direction of fluid flow in accordance with a representative embodiment. Various aspects and details of the various representative embodiments described in connection with FIGS. 1A-2A are common to the description of FIG. 2B, and may not be repeated in order to avoid obscuring the description of FIG. 2B.

As shown, the helical flow modifier 200 alters the shape of the vein or artery in the region it is deployed, beneficially resulting in substantially helical flow of fluid 206 through the damaged portion of the vein or artery. Accordingly, the helical flow modifier 200 recanalizes the vessels of the deep venous system or peripheral arteries to the foster substantially helical flow in damaged or diseased portions of the veins and arteries when deployed therein. As such, and as noted above, this substantially helical flow of fluid 206 resembles the normal helical flow of fluid in undamaged diseased (i.e., “normal”) veins and arteries. As such, and like in normal arteries and veins, the resultant helical flow limits flow instability and represents natural optimization of fluid transport in the cardiovascular system.

FIG. 2C is a perspective view of the helically shaped insert (shown in cut away) of the helical flow modifier of FIG. 2A. Various aspects and details of the various representative embodiments described in connection with FIGS. 1A-2B are common to the description of FIG. 2C, and may not be repeated in order to avoid obscuring the description of FIG. 2C.

Again, the helical flow modifier 200 comprises a shape that fosters substantially helical fluid flow through the portion of the artery or vein where the helical flow modifier 200 is disposed. The helically shaped stent 204 is attached to the inner surface of the portion of the artery or vein where the helical flow modifier 200 is disposed. As described above, the helical flow modifier 200 is deployed in a vein or artery with a catheter or similar device in an initially compressed state. Upon deployment to a desired location, the catheter is removed. The helical flow modifier 200 is designed to expand to it original shape and dimension and is force-fit against the inner surface of the portion of the vein or artery in which it is deployed. As such, the helically shaped stent 204 has dimensions selected to provide an outward radial (x-direction in the coordinate system of FIG. 2A) force to the inner surface of the portion of the vein or artery in which the helical flow modifier 100 is deployed.

FIG. 2D is a top view of the helical flow modifier 200 of FIG. 2A looking in the −y direction and in the y-z plane according to the coordinate system of FIG. 2A.

Again, and as noted above in connection with FIG. 1D, FIG. 2D illustrates that the helical flow modifier 200 does not substantially obstruct the intraluminal space so that fluid (e.g., blood) flow through the region of the lumen where the helical flow modifier 200 is deployed

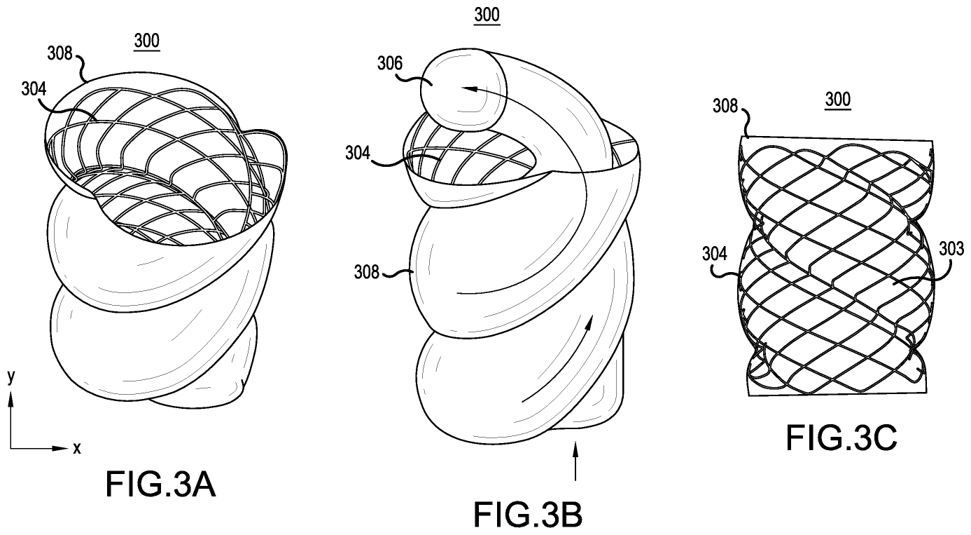

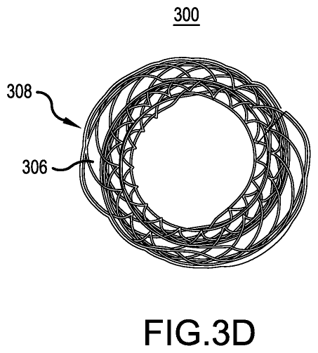

FIG. 3A is a perspective view of a helical flow modifier 300 in accordance with a representative embodiment. Various aspects and details of the various representative embodiments described in connection with FIGS. 1A-1E are common to the description of FIG. 3A, and may not be repeated in order to avoid obscuring the description of FIG. 3A.

The helical flow modifier 300 is adapted to be disposed into a lumen (not shown in FIG. 3A), as is adapted to alter a flow characteristic of a fluid in the lumen along a helical trajectory. In the representative embodiment of FIG. 3A, the helical flow modifier 300 comprises a helically shaped stent 304 adapted to be disposed in the lumen. In one representative embodiment, the helical flow modifier 300 is attached to an inner surface (alternatively referred to as an inner wall) of the vein or artery in which it is deployed. In this representative embodiment, the helical flow modifier 300 comprises nitinol or other similar shape memory metal or other material. Like the helical flow modifier 100 described above, the helical flow modifier 300 is deployed in a vein or artery with a catheter or similar device in an initially compressed state. Upon deployment to a desired location, the catheter is removed. The helical flow modifier 300 is designed to expand to it original shape and dimension and is force-fit against the inner surface vein or artery. As such, the helical flow modifier 300 has dimensions selected to provide an outward radial (x-direction in the coordinate system of FIG. 3A) force to the inner surface of the portion of the vein or artery in which the helical flow modifier 300 is deployed.

The helical flow modifier 300 also comprises a cover 308 or sheath disposed on its outer surface as shown. The cover 308 beneficially keep the fluid flow within the inside lumen of the helically shaped stent 304 of the helical flow modifier 300, rather than allowing flow to pass through the braid of the helically shaped stent. This is especially beneficial when there is a slight gap between the helical pattern of the helically shaped stent 304 and the inner wall of the vein or artery.

In accordance with a representative embodiment, the cover 308 comprises one of a number of known suitably materials for its function. These include, but are not limited to synthetic polymers such as Polytetrafluoroethylene (PTFE), Ultra High Molecular Weight Polyethylene (UHMWPE) or high density polyethylene. The material selected for the cover 408 should be anti-thrombotic in order not to promote thrombus formation in the implant.

Notably, the helical flow modifier 300 has a substantially helical shape, which is more clearly shown in FIGS. 3B and 3C, and provides a helical support along the inner surface portion of the vein or artery in which the helical flow modifier 300 is deployed. This helical support in turn provides a shape to the inner surface of the region of the vein or artery in which the helical flow modifier 300 is deployed that promotes substantially helical flow. As such, when deployed in the portion of vein or artery, the helical flow modifier 300 provides a shape to the vein or artery lumen in which the helical flow modifier 300 is deployed, resulting in substantially helical flow through the artery or vein where the helical flow modifier is deployed. Beneficially, the helical flow modifier 300 is deployed in a diseased or otherwise damaged portion of the vein or artery in which it is deployed.

FIG. 3B is a perspective view of the helical flow modifier 300 of FIG. 3A showing the direction of fluid flow in accordance with a representative embodiment. Various aspects and details of the various representative embodiments described in connection with FIGS. 1A-3A are common to the description of FIG. 3B, and may not be repeated in order to avoid obscuring the description of FIG. 3B.

As shown, the helical flow modifier 300 alters the shape of the vein or artery in the region it is deployed, beneficially resulting in substantially helical flow of fluid 306 through the damaged portion of the vein or artery. Accordingly, the helical flow modifier 300 recanalizes the vessels of the deep venous system or peripheral arteries to the foster substantially helical flow in damaged or diseased portions of the veins and arteries when deployed therein. As such, and as noted above, this substantially helical flow of fluid 306 resembles the normal helical flow of fluid in undamaged diseased (i.e., “normal”) veins and arteries. As such, and like in normal arteries and veins, the resultant helical flow limits flow instability and represents natural optimization of fluid transport in the cardiovascular system.

FIG. 3C is perspective view of the helically shaped insert (shown in cut away) of the helical flow modifier 300 of FIG. 3A. Various aspects and details of the various representative embodiments described in connection with FIGS. 3A-3B are common to the description of FIG. 3C, and may not be repeated in order to avoid obscuring the description of FIG. 3C.

Again, the helical flow modifier 300 comprises a shape that fosters substantially helical fluid flow through the portion of the artery or vein where the helical flow modifier 300 is disposed. The helically shaped stent 304 is attached to the inner surface of the portion of the artery or vein where the helical flow modifier 300 is disposed. As described above, the helical flow modifier 300 is deployed in a vein or artery with a catheter or similar device in an initially compressed state. Upon deployment to a desired location, the catheter is removed. The helical flow modifier 300 is designed to expand to it original shape and dimension and is force-fit against the inner surface of the portion of the vein or artery in which it is deployed. As such, the helically shaped stent 304 has dimensions selected to provide an outward radial (x-direction in the coordinate system of FIG. 3A) force to the inner surface of the portion of the vein or artery in which the helical flow modifier 100 is deployed.

FIG. 3D is a top view of the helical flow modifier of FIG. 3A looking in the −y direction and in the y-z plane according to the coordinate system of FIG. 3A.

Again, and as noted above in connection with FIGS. 1D and 2D, FIG. 2D illustrates that the helical flow modifier 200 does not substantially obstruct the intraluminal space so that fluid (e.g., blood) flow through the region of the lumen where the helical flow modifier 200 is deployed

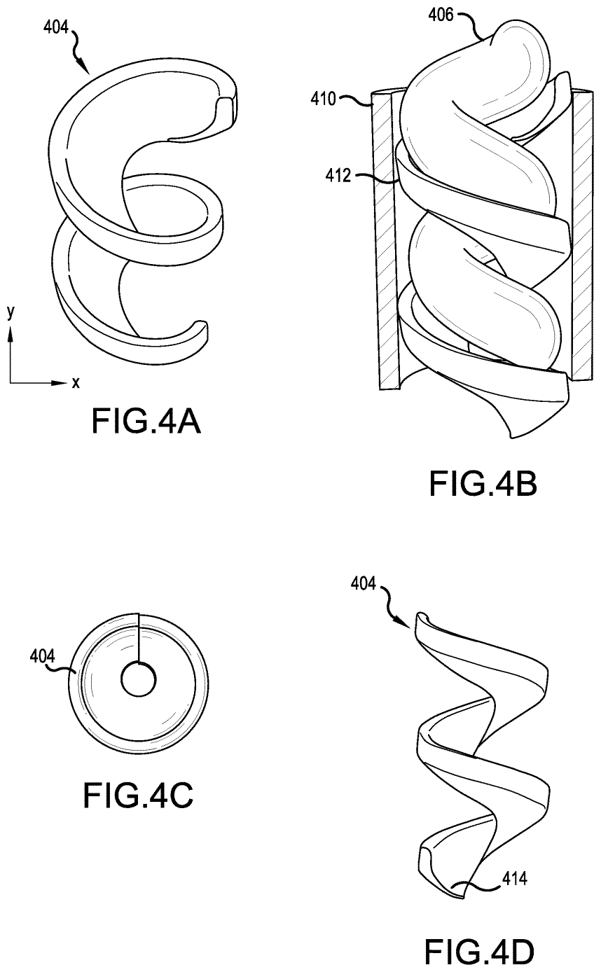

FIG. 4A is a perspective view of a helical flow modifier 400 in accordance with a representative embodiment. Various aspects and details of the various representative embodiments described in connection with FIGS. 1A-3D are common to the description of FIG. 4A, and may not be repeated in order to avoid obscuring the description of FIG. 4A.

The helical flow modifier 400 comprises helically shaped insert 404. Unlike the helical flow modifiers of the representative embodiments described above, the helical flow modifier 400 is not disposed in a stent, but rather, as described in connection with FIG. 4B is inserted directly into the vein or artery in a portion of the vein or artery that is diseased or otherwise damaged.

The helically shaped insert 404 comprises a material suitable for deployment in the body. The helically shaped insert is illustratively a nitinol metal alloy or other suitable biocompatible material that will maintain the coil shape when deployed inside the vessel, having outward force to position it against the vessel wall.

The helical flow modifier 400 is adapted to be disposed into a lumen (not shown in FIG. 4A), as is adapted to alter a flow characteristic of a fluid in the lumen along a helical trajectory. In the representative embodiment of FIG. 4A, the helical flow modifier 400 comprises the helically shaped insert 404, which serves as a guide for fluid to move through the portion of the artery or vein where the helically shaped insert. In one representative embodiment, the helically shaped insert is attached to an inner surface of the artery or vein (see FIG. 4B) and because of its shape functions to create a path for helical flow in the portion of the artery or vein in which it is disposed.

Like the helical flow modifiers described above, the helical flow modifier 400 is deployed in a vein or artery with a catheter or similar device. Alternatively, the helical flow modifier 400 may be implanted surgically. For example, the helical flow modifier 400 could be surgically implanted in distal portions of the deep venous system, which may be accessed for example, during an endophlebectomy procedure. However, unlike the helical flow modifiers 100, 200, 300 described above, the helical flow modifier 400 does not foster helical flow by altering the shape of the portion of the vein or artery where it is deployed, but rather fosters helical flow because of its shape.

FIG. 4B is a perspective view of the helically shaped insert 404 of FIG. 4A deployed in a lumen 410 (shown in cut away) illustrating the direction of fluid flow 406 in accordance with a representative embodiment. Various aspects and details of the various representative embodiments described in connection with FIGS. 1A-4A are common to the description of FIG. 4B, and may not be repeated in order to avoid obscuring the description of FIG. 4B.

As shown in FIG. 4B, the helical flow modifier 400 has surfaces 412 that are adapted to abut inner surfaces 411 of the lumen 410 (e.g., an artery or vein). Specifically, the apposition of the surfaces 412 and the inner surfaces 411 of the lumen 410 are designed to reduce the contact area between the helically shaped insert 404 and the inner surface 411 of the lumen 410. Notably, the helically shaped insert 404 has a flattened area compared to the cylindrical wire shape of the helical flow modifiers described above, for example. This flattened area helps the helically shaped insert 404 to engage the vessel wall and substantially prevent migration or movement. The material selected for the helically shaped insert 404 provides a constant outward force, opposing the vessel wall. This force holds the helically shaped insert in place and substantially prevents migration.

As described more fully below, the helically shaped insert 404 has a cross-sectional shape that beneficially reduces or substantially eliminates retrograde flow of fluid in the direction opposite the direction of fluid flow 406. Accordingly, and as will be appreciated by one of ordinary skill in the art having had the benefit of the present disclosure, a separate valve may not be required when the helically shaped insert 404 is deployed since retrograde flow is substantially avoided.

FIG. 4C is a top view of the helical flow modifier of FIG. 4A looking in the −y direction and in the y-z plane according to the coordinate system of FIG. 4A.

The helically shaped insert 404 is adapted to be compressed into a catheter and when deployed, it takes a shape that expands and opposes the vessel wall.

FIG. 4D is a perspective view of the helical flow modifier showing a shape of the helical structure designed to resist retrograde flow (flow in the opposite direction of flow shown in FIG. 4B) in accordance with a representative embodiment. Various aspects and details of the various representative embodiments described in connection with FIGS. 1A-4C are common to the description of FIG. 4B, and may not be repeated in order to avoid obscuring the description of FIG. 4D.

As alluded to above, the helically shaped insert 404 comprises a cross-sectional shape that beneficially reduces or substantially eliminates retrograde flow of fluid in the lumen 410. Specifically, the helically shaped insert 404 has a shelf 414 that will help orient forward flow into a helical orientation and also provide a physical barrier to prevent or reduce reflux by acting as a “non-mobile” partial valve. Moreover, although forward flow is augmented by helical nature of movement “up” the helical flow modifier 400, the helically shaped insert 404 has a slightly reduced lumen diameter (see FIG. 4C) at its upper end, so the column of blood will be less able to move in a reflux or reverse manner given the smaller lumen from the longitudinal plane.

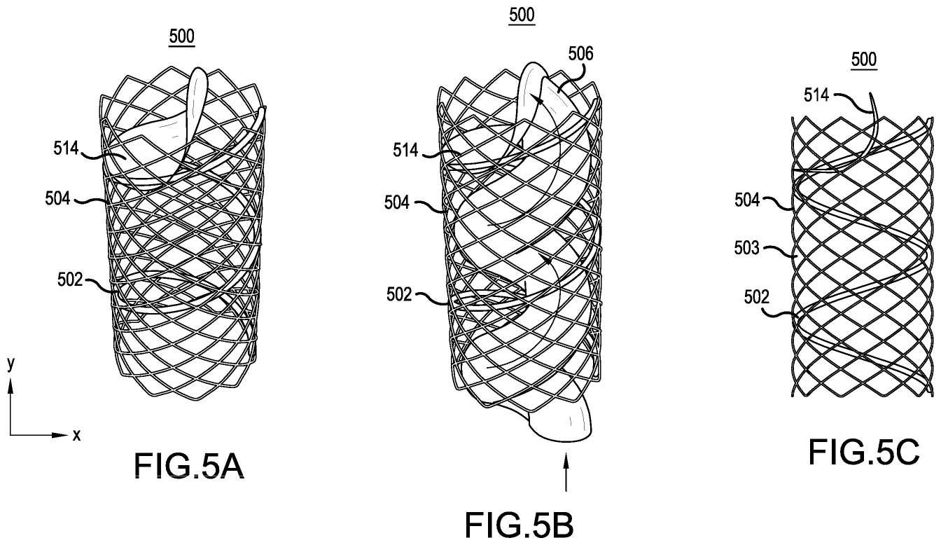

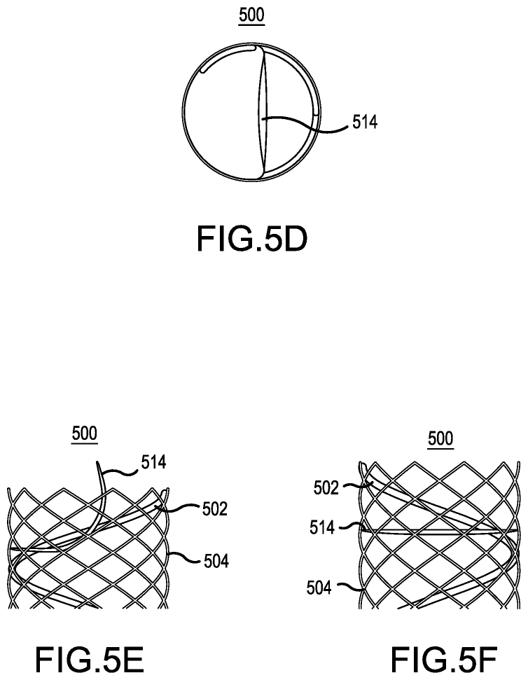

FIG. 5A is a perspective view of a helical flow modifier 500 in accordance with a representative embodiment. Various aspects and details of the various representative embodiments described in connection with FIGS. 1A-4D are common to the description of FIG. 5A, and may not be repeated in order to avoid obscuring the description of FIG. 5A.

The helical flow modifier 500 is adapted to be disposed into a lumen (not shown in FIG. 5A), as is adapted to alter a flow characteristic of a fluid in the lumen along a helical trajectory. Notably, and as described more fully below, the helical flow modifier 500 comprises a monocuspid valve 514 disposed therein. Notably, as shown, the monocuspid valve 514 is in an open state. When in a closed state, the monocuspid valve 514 would cover the cross-sectional area of the helical flow modifier 500. It is noted that the monocuspid valve 514 is not limited to use in helical flow modifiers having the helically shaped element 502. More generally, the monocuspid valve 514 may be deployed in helical flow modifiers 200, 300 to deploy the monocuspid valve 514 in a portion of an artery or vein to regulate fluid flow in one direction when open, and limit retrograde fluid flow, such as described in connection with FIG. 5B.

In the representative embodiment of FIG. 5A, the helical flow modifier 500 comprises a helically shaped element 502 disposed in a stent 504 adapted to be disposed in the lumen. In one representative embodiment, the helically shaped element 502 is attached to an inner surface 503 (alternatively referred to as an inner wall) of the stent 504. Alternatively, the helically shaped element 502 may be interwoven in the braid of the stent 504 to alter the shape of the stent 504 to foster helical blood flow as described more fully below.

In this representative embodiment, the helically shaped element 502 comprises nitinol or other similar shape memory metal or other material. As described more fully below, the helical flow modifier 500 is deployed in a vein or artery with a catheter or similar device in an initially compressed state. Upon deployment to a desired location, the catheter is removed. The helically shaped element 502 is designed to expand to it original shape and dimension and is force-fit against the inner surface 503 of the stent 504. As such, the helically shaped element 502 has dimensions selected to provide an outward radial (x-direction in the coordinate system of FIG. 5A) force to the inner surface 503 of the stent 504, and therefore to the inner surface of the portion of the vein or artery in which the helical flow modifier 500 is deployed.

Notably, once deployed the helically shaped element 502 provides a helical skeleton or support along the inner surface 503 of the stent 504. This helical skeleton or support in turn provides a shape to the region of the vein or artery in which the helical flow modifier 500 is deployed that promotes substantially helical flow. Alternatively, as noted above, the helically shaped element 502 may be interwoven in the braid of the stent 504 to alter the shape of the stent 504 to foster helical blood flow as described more fully below.

Illustratively, when deployed in the vein or artery, the helically shaped element 502 provides a helical shape to the vein or artery in which the helical flow modifier 500 is deployed, resulting in substantially helical flow through the artery or vein where the helical flow modifier is deployed. Beneficially, the helical flow modifier 500 is deployed in a diseased or otherwise damaged portion of the vein or artery in which it is deployed.

In accordance with a representative embodiment, the helically shaped element 502 comprises a suitable biocompatible material, such as nitinol, in the shape of a wire Specifically, the wire used for the helically shaped element as a cylindrical cross section having dimensions selected to provide the force-fit and shape to alter the flow of fluid through the damaged portion of the vein or artery where it is deployed.

FIG. 5B is a perspective view of the helical flow modifier 500 of FIG. 5A showing the direction of fluid flow in accordance with a representative embodiment. Various aspects and details of the helical flow modifier 500 described in connection with FIG. 5A are of course common to the description of FIG. 5B, and may not be repeated in order to avoid obscuring the description of FIG. 5B.

As shown, the helical flow modifier 500 alters the shape of the vein or artery in the region it is deployed, beneficially resulting in substantially helical flow of fluid 506 through the damaged portion of the vein or artery. Accordingly, the helical flow modifier 500 recanalizes the vessels of the deep venous system or peripheral arteries to the foster substantially helical flow in damaged or diseased portions of the veins and arteries when deployed therein. As such, and as noted above, this substantially helical flow of fluid 506 resembles the normal substantially helical flow of fluid in undamaged diseased (i.e., “normal”) veins and arteries. As such, and like in normal arteries and veins, the resultant substantially helical flow limits flow instability and represents natural optimization of fluid transport in the cardiovascular system.

Moreover, the monocuspid valve 514 disposed in the helical flow modifier 500 is in shown an open state. When in a closed state, the monocuspid valve 514 would cover the cross-sectional area of the helical flow modifier 500 and regulates fluid flow in one direction (helical flow of fluid 506) when open, and limit retrograde fluid flow, such as described in connection with FIG. 5B.

The helically shaped insert 404 may also have a valve (not shown in FIG. 4B) attached thereto. The position of the valve may be angled such that the anchored section is tilted down from the section that opens, such that when flow stops, the column of fluid back flows toward the anchored section of the valve, preventing retrograde flow. The concave shape and leaflet position aims to resist prolapse of the leaflet and mimics the shape of the venous sinus pocket that is characteristic of healthy venous valve. This shape may also promote flow to circulate within the valve cavity to resist flow stagnation that may propagate venous disease. Further details of the promotion of flow to circulate within the valve and resist stagnation may be found in Hajati, et al. (“Fluid-Structure Interaction of Blood Flow Around a Vein Valve” BioImpacts, (2020), 10(3), 169-175).

FIG. 5C is a perspective view (shown in cut-away) of the helical flow modifier 500 of FIG. 5A. Various aspects and details of the helical flow modifier 500 described in connection with FIGS. 5A and 5B are of course common to the description of FIG. 5C, and may not be repeated in order to avoid obscuring the description of FIG. 5C.

Again, the helical flow modifier 500 comprises the helically shaped element 502 disposed in the stent 504 adapted to be disposed in the lumen. In one representative embodiment, the helically shaped element 502 is attached to the inner surface 503 (in this case in the rear inner wall of the stent 504) of the stent 504 as described above. As described more fully below, the helical flow modifier 500 is deployed in a vein or artery with a catheter or similar device in an initially compressed state. Upon deployment to a desired location, the catheter is removed. The helically shaped element 502 is designed to expand to it original shape and dimension and is force-fit against the inner surface 503 of the stent. As such, the helically shaped element 502 has dimensions selected to provide an outward radial (x-direction in the coordinate system of FIG. 5A) force to the inner surface 503 of the stent 504, and therefore to the inner surface of the portion of the vein or artery in which the helical flow modifier 500 is deployed.

FIG. 5D is a top view of the helical flow modifier of FIG. 5A looking in the −y direction and in the y-z plane according to the coordinate system of FIG. 5A. [As shown, the monocuspid valve 514 is in an open state. When in a closed state, the monocuspid valve 514 would cover the cross-sectional area of the helical flow modifier 500. It is noted that the monocuspid valve 514 is not limited to use in helical flow modifiers having the helically shaped element 502. More generally, the monocuspid valve 514 may be deployed in helical flow modifiers 200, 300, 400 to deploy the monocuspid valve 514 in a portion of an artery or vein to regulate fluid flow in one direction when open, and limit retrograde fluid flow, such as described in connection with FIG. 5B.

FIG. 5D is a side view of the helical flow modifier having the monocuspid valve 514 in an open state. As will be appreciated, helical flow of fluid 506 in the direction noted in FIG. 5B occurs.

FIG. 5E is a side view of the helical flow modifier having the monocuspid valve 514 in a closed state. As will be appreciated, fluid flow in a direction opposite to that shown in FIG. 5B (the −y direction according to the coordinate system of FIG. 5A) is substantially prevented. So, for example, in a cardiovascular application, the monocuspid valve 514 is open when the heart is beating, and closed between beats of the heart.

Finally, like certain helical flow modifiers described above, the helical flow modifier 500 may be deployed the catheter or similar device (not shown in FIGS. 5A-5D) in an initially compressed state. Upon deployment to a desired location, the catheter is removed. In the presently described representative embodiment, the helically shaped element 502 is designed to expand to it original shape and dimension and is force-fit against the inner surface 503 of the stent. Alternatively, in embodiments in which the helically shaped element 502 is interwoven in the stent, when deployed, the stent 504 expands to its original helical shape, and dimension. As such, the helically shaped element 502 has dimensions selected to provide an outward radial (x-direction in the coordinate system of FIG. 5A) force to the inner surface 503 of the stent 504, and therefore to the inner surface of the portion of the vein or artery in which the helical flow modifier 500 is deployed.

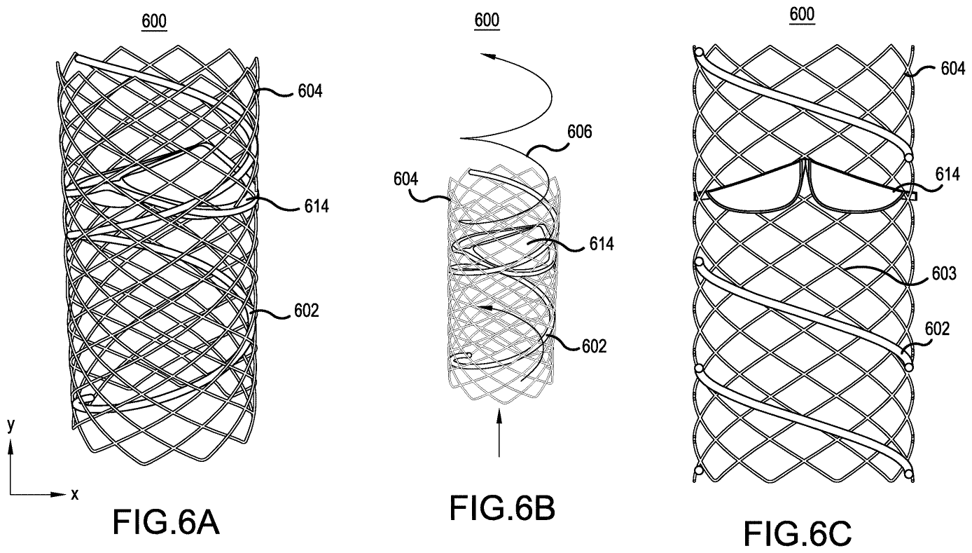

FIG. 6A is a perspective view of a helical flow modifier 600 in accordance with a representative embodiment. Various aspects and details of the various representative embodiments described in connection with FIGS. 1A-5E are common to the description of FIG. 6A, and may not be repeated in order to avoid obscuring the description of FIG. 6A.

The helical flow modifier 600 is adapted to be disposed into a lumen (not shown in FIG. 6A), as is adapted to alter a flow characteristic of a fluid in the lumen along a helical trajectory. Notably, and as described more fully below, the helical flow modifier 600 comprises a bicuspid valve 614 disposed therein. Notably, as shown, the bicuspid valve 614 is in a closed state, and covers the cross-sectional area (in the x-z plane of FIG. 6A) of the helical flow modifier 600. In an open state, the bicuspid valve moves to allow fluid flow in a desired dimension. It is noted that the bicuspid valve 614 is not limited to use in helical flow modifiers having the helically shaped element 602. More generally, the bicuspid valve 614 may be deployed in helical flow modifiers 200, 300 to deploy the bicuspid valve 614 in a portion of an artery or vein to regulate fluid flow in one direction when open, and limit retrograde fluid flow, such as described in connection with FIG. 6B.

In the representative embodiment of FIG. 6A, the helical flow modifier 600 comprises a helically shaped element 602 disposed in a stent 604 adapted to be disposed in the lumen. In one representative embodiment, the helically shaped element 602 is attached to an inner surface 603 (alternatively referred to as an inner wall) of the stent 604 as described above. Alternatively, the helically shaped element 602 may be interwoven in the braid of the stent 604 to alter the shape of the stent 604 to foster helical blood flow as described more fully below.

In this representative embodiment, the helically shaped element 602 comprises nitinol or other similar shape memory metal or other material. As described more fully below, the helical flow modifier 600 is deployed in a vein or artery with a catheter or similar device in an initially compressed state. Upon deployment to a desired location, the catheter is removed. The helically shaped element 602 is designed to expand to it original shape and dimension and is force-fit against the inner surface 603 of the stent 604. As such, the helically shaped element 602 has dimensions selected to provide an outward radial (x-direction in the coordinate system of FIG. 6A) force to the inner surface 603 of the stent 604, and therefore to the inner surface of the portion of the vein or artery in which the helical flow modifier 600 is deployed.

Notably, once deployed the helically shaped element 602 provides a helical skeleton or support along the inner surface 603 of the stent 604. This helical skeleton or support in turn provides a shape to the region of the vein or artery in which the helical flow modifier 600 is deployed that promotes substantially helical flow. Alternatively, as noted above, the helically shaped element 602 may be interwoven in the braid of the stent 604 to alter the shape of the stent 604 to foster helical blood flow as described more fully below.

Illustratively, when deployed in the vein or artery, the helically shaped element 602 provides a helical shape to the vein or artery in which the helical flow modifier 600 is deployed, resulting in substantially helical flow through the artery or vein where the helical flow modifier is deployed. Beneficially, the helical flow modifier 600 is deployed in a diseased or otherwise damaged portion of the vein or artery in which it is deployed.

In accordance with a representative embodiment, the helically shaped element 602 comprises a suitable biocompatible material, such as nitinol, in the shape of a wire. Specifically, the wire used for the helically shaped element as a cylindrical cross section having dimensions selected to provide the force-fit and shape to alter the flow of fluid through the damaged portion of the vein or artery where it is deployed.

FIG. 6B is a perspective view of the helical flow modifier 600 of FIG. 6A showing the direction of fluid flow in accordance with a representative embodiment. Various aspects and details of the helical flow modifier 600 described in connection with FIG. 6A are of course common to the description of FIG. 6B, and may not be repeated in order to avoid obscuring the description of FIG. 6B.

As shown, the helical flow modifier 600 alters the shape of the vein or artery in the region it is deployed, beneficially resulting in substantially helical flow of fluid 606 through the damaged portion of the vein or artery. Accordingly, the helical flow modifier 600 recanalizes the vessels of the deep venous system or peripheral arteries to the foster substantially helical flow in damaged or diseased portions of the veins and arteries when deployed therein. As such, and as noted above, this substantially helical flow of fluid 606 resembles the normal substantially helical flow of fluid in undamaged diseased (i.e., “normal”) veins and arteries. As such, and like in normal arteries and veins, the resultant substantially helical flow limits flow instability and represents natural optimization of fluid transport in the cardiovascular system.

Moreover, the bicuspid valve 614 disposed in the helical flow modifier 600 is in shown the closed state, with its two leaflets down. When in a closed state, the bicuspid valve 614 would cover the cross-sectional area of the helical flow modifier 600 and regulates fluid flow in one direction (helical flow of fluid 606) and limit retrograde fluid flow (−y axis in the coordinate system of FIG. 6A).

FIG. 6C is a perspective view (shown in cut-away) of the helical flow modifier 600 of FIG. 6A. Various aspects and details of the helical flow modifier 600 described in connection with FIGS. 6A and 6B are of course common to the description of FIG. 6C, and may not be repeated in order to avoid obscuring the description of FIG. 6C.

Again, the helical flow modifier 600 comprises a helically shaped element 602 disposed in a stent 604 adapted to be disposed in the lumen. In one representative embodiment, the helically shaped element 602 is attached to the inner surface 603 (in this case in the rear of the stent 604) inner wall) of the stent 604. As described more fully below, the helical flow modifier 600 is deployed in a vein or artery with a catheter or similar device in an initially compressed state. Upon deployment to a desired location, the catheter is removed. The helically shaped element 602 is designed to expand to it original shape and dimension and is force-fit against the inner surface 603 of the stent. As such, the helically shaped element 602 has dimensions selected to provide an outward radial (x-direction in the coordinate system of FIG. 6A) force to the inner surface 603 of the stent 604, and therefore to the inner surface of the portion of the vein or artery in which the helical flow modifier 600 is deployed.

In an open state (not shown), the bicuspid valve 614 opens at its center with its two leaflets being moved upward like monocuspid valve 514 except with two leaflets adapted to open in the center of the stent 604.

The present disclosure, through one or more of its various aspects, embodiments and/or specific features or sub-components, is thus intended to bring out one or more of the advantages as specifically noted below.

The apparatuses, systems and methods of the present teachings may be used in a variety of non-invasive medical procedures and with a variety of other apparatuses and systems where deployment in a venous or arterial system, is desired.

Although apparatuses, systems and methods including first and second memory wires are described with reference to several exemplary embodiments, it is understood that the words that have been used are words of description and illustration, rather than words of limitation. Changes may be made within the purview of the appended claims, as presently stated and as amended, without departing from the scope and spirit of additive manufacturing of a medical stent based on a computational modeled outcome prediction in its aspects.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “teachings” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An apparatus comprising:
a helical flow modifier adapted to be disposed into a lumen, wherein the helical flow modifier alters a flow characteristic of a fluid in the lumen along a helical trajectory.

2. The apparatus of claim 1, wherein the helical flow modifier comprises a helically shaped element disposed in a stent.

3. The apparatus of claim 2, wherein the helically shaped element comprises a wire disposed along an inner surface of the stent.

4. The apparatus of claim 3, further comprising a valve disposed in the stent in a region of the helical flow modifier.

5. The apparatus of claim 4, wherein the valve is a bicuspid valve or a monocuspid valve.

6. The apparatus of claim 1, wherein the helical flow modifier comprises a stent having a helically shaped outer wall.

7. The apparatus of claim 6, further comprising a valve disposed in the stent in a region of the helical flow modifier.

8. The apparatus of claim 7, wherein the valve is a bicuspid valve or a monocuspid valve.

9. The apparatus of claim 7, further comprising a cover layer disposed on an outer surface of the stent.

10. The apparatus of claim 1, wherein the helical flow modifier is not disposed in a stent.

11. The apparatus of claim 10, wherein the helical flow modifier comprises a helically shaped insert.

12. The apparatus of claim 11, wherein one end of the helical shaped insert has a first end and a second end, and the second end is adapted to resist retrograde flow of a fluid.

13. A method of delivering an apparatus, the method comprising:
disposing a helical flow modifier into a lumen, wherein the helical flow modifier alters a flow characteristic of a fluid in the lumen along a helical trajectory.

14. The method of claim 13, wherein the method further comprises disposing the helical shaped modifier in a stent and disposing the stent in a catheter to locate the stent and the helical shaped modifier in a particular location of the lumen.

15. The method of claim 14, wherein the method further comprises disposing a valve in the stent in a region of the helical flow modifier.

16. The method of claim 13, wherein the helical flow modifier comprises a stent having a helically shaped outer wall.

17. The method of claim 16, wherein the method further comprises disposing a valve in the stent in a region of the helical flow modifier.

18. The method of claim 13, wherein the helical flow modifier is not disposed in a stent.

19. The method of claim 18, wherein one end of the helical flow modifier has a first end and a second end, and the second end is adapted to resist retrograde flow of a fluid.
